(19) United States
(12) Patent Application Publication
(10) Pub. No.: US 2024/0115465 A1
(43) Pub. Date: Apr. 11, 2024

(54) SYSTEM AND METHOD FOR PLACEMENT VERIFICATION OF NASOGASTRIC TUBE

(71) Applicant: Mary Hitchcock Memorial Hospital, for itself and on behalf of Dartmouth-Hitchcock Clinic, Labanon, NH (US)

(72) Inventors: Tyler K. Hartman, Lebanon, NH (US); Megan Clark, Norwich, VT (US); Parveer Kaur, Spokane Valley, WA (US); Kent H. Campbell, Boston, MA (US); Shannon Kossmann, Santa Fe, NM (US); Jacob R. Rozak, Medway, MA (US)

(21) Appl. No.: 18/223,108

(22) Filed: Jul. 18, 2023

Related U.S. Application Data
(60) Provisional application No. 63/390,351, filed on Jul. 19, 2022.

(51) Int. Cl. A61J 15/00

(52) U.S. Cl. CPC A61J 15/0026; A61J 15/0003

(57) ABSTRACT

A system and method for verification of nasogastric tube (NGT) placement is provided. A fiber optic light guide includes a proximal end with a light source connection and a distal end having a light emitting tip. The light guide is constructed and arranged for insertion into a lumen of the NGT. A stop that prevents the distal end of the light guide from exiting a distal end of the NGT. The stop is adapted to engage a connector on the proximal end of the NGT. The light emitting tip can comprises a radial emitter. The light source can emit light at approximately 650 nm with an output power of approximately 30W. The stop is located so that the distal end of the light guide is positioned at an offset from the distal end of the NGT when the stop engages the proximal end of the NGT.

### RELATED APPLICATION

This application claims the benefit of co-pending U.S. Provisional Application Ser. No. 63/390,351, entitled SYSTEM AND METHOD FOR PLACEMENT VERIFICATION OF NASOGASTRIC TUBE, filed Jul. 19, 2022, the teachings of which are expressly incorporated herein by reference.

### FIELD OF THE INVENTION

This invention relates to nasogastric tubes (NGTs), and more particular to NGTs and methods for placement thereof in infants.

### BACKGROUND OF THE INVENTION

Over 90% of infants admitted into the NICU require nasogastric feeding tubes (NGTs) to receive nutrients they are unable to eat or drink by mouth. NGT insertion is a common procedure where a flexible tube is fed into a patient's nose, down their esophagus, and into their stomach. Before placement, the practitioner measures the expected inserted length of the NGT based on the patient's anatomical features. The nurse then inserts the NGT by placing it in the patient's nose and pushing it straight back towards the patient's posterior pharynx, stopping at the expected length. During NGT insertion, tube misplacement can occur in several different ways. Placement in the lung is most dangerous, with the potential of pneumothorax or death.

More generally, insertion of an NGT is a high-volume practice commonly performed by nurses and other practitioners as a blind procedure, without the use of technology to guide or visualize the path of the tube. As shown in FIG. 1, successful NGT placement consists of insertion of a flexible, biocompatible tube into a patient's nose, down their esophagus and into their stomach. Babies may rely on NGTs for feeding for about 3 months before transitioning to bottle and breastfeeding. NGTs are replaced very frequently: up to multiple times a week. Misplacements are possible but since there is no national reporting system in the U.S., it is very difficult to quantify the number of NGTs placed and the subsequent number of misplacements. Research has demonstrated that 10.2% of misplacements occurred in the infant population, under the age of 1. A shown in FIG. 1, depicting the exemplary anatomy of an infant 100 with NGT 110 placed therein. In general, there are five key types of misplacement. From most to least common these are esophageal placement and/or curling in the esophagus 120, post-pyloric placement 130, perforation of the esophagus 140, or lung misplacement 150—as opposed to normal stomach (160) placement as shown. All three types of misplacements concerning the esophagus pose risk of aspiration (seen in less than 1 in 200 NGT placements). Lung misplacements are the rarest, but most dangerous, type of misplacement—with a 30-50% mortality rate. Intensive Care Nursery (ICN) survey data and health care provider interviews demonstrate that misplacements are not correlated with provider expertise, provider level of education or provider fatigue. With the exception of esophageal misplacements, it is evident that misplacements are due to random events. Esophageal misplacements are viewed as a common secondary problem because this misplacement is a result of the tube being tugged by the infant.

Due to the uncertainty of NGT placement (when performed as a blind procedure) and the potential for aspiration or mortality if placed incorrectly, most hospitals require some form of tube placement verification. Existing methods of verification are either inaccurate (e.g. pH testing of gastric secretions) or expensive, and potentially unsafe (e.g. x-ray), especially given the frequency of NGT placement in neonates. Based on the frequency of NGT placements, the shortcomings of state-of-the-art verification methods, and lack of a standardized placement verification method, the need for a technological innovation that ensures correct tube placement is clear and long-felt.

Since the NGT placement procedure has no visual or haptic feedback, misplacements can occur without the knowledge of the healthcare provider and occur randomly (no correlation with experience). Due to the uncertainty of tube placement and the potential for catastrophic consequences if placed incorrectly, most hospitals require some form of tube placement verification. Existing methods of verification are inaccurate, expensive, or unsafe for neonates, especially at the frequency at which NGT are placed. The regularity of NGT placements and the shortcomings of current verification methods substantiate the need for a low cost technological innovation that ensures correct tube placement efficiently, effectively, and safely.

It is desirable to provide a system and method for verifying placement of the NGT in a manner that does not alter its current parameters or general procedures for insertion and/or use.

### SUMMARY OF THE INVENTION

This invention overcomes disadvantages of the prior art by providing a fiber optic cable with a side-firing tip that can be inserted down the lumen of the NGT before the NGT is placed, stopping approximately 0.5 cm from the tube's distal end. After the preloaded tube is inserted into the patient, a quick-connect system would allow the medical practitioner to connect a (e.g.) 650 nm, 30 mW light source to the proximal end of the fiber allowing visual confirmation of the NGT placement based on the light penetrating through the patient's abdomen from the tube's distal end. After verification, the fiber insert is removed and discarded prior to feeding whereas the light source would be a re-chargeable, hand-held device. Any potential solution requires minimal changes to the properties of the NGT. The NGT should be 6.5 French diameter, so as to ensure placement will be non-traumatic by maintaining the highly flexible nature of the tube, the minimal weight, biocompatible material, and preserve the functional ability of the tube to deliver feed to the patient. The NGT, and any other components that come into contact with the patient, should also be either disposable or sterilizable in order to be safe to use.

While misplacement into the lung is the least common type of NGT misplacement, it is the most dangerous and is often a primary concern for healthcare providers. In order to ensure the NGT is in the stomach rather than in the lung, the system and method herein should be able to determine placement of the distal end of the NGT tube to within at least 1.5 cm within a patient's anatomy. For accuracy the system should enable the correct determination of NGT placement at least 95% of the time and last for at least 5 years to be comparable in durability to other devices frequently used in the ICN.

In an illustrative embodiment a system for verification of nasogastric tube (NGT) placement is provided. It includes a fiber optic light guide having a proximal end with a light source connection and a distal end having a light emitting tip. The light guide can have a diameter and a length constructed and arranged for insertion into a lumen of the NGT. A stop/stopper prevents the distal end of the light guide from exiting a distal end of the NGT. Illustratively, the stop is adapted to engage a connector on the proximal end of the NGT and/or the light emitting tip comprises a radial emitter. The light source connection can comprise an LC connector that removably engages a handheld, switchable light source. The light source can emit light in a range of approximately 600-700 nm, and more particularly at approximately 650 nm, which provides good visibility through tissue for a wide range of skin tomes (melanin content). The light source can emit light with an output power of approximately 30W. The stop can be located so that the distal end of the light guide is positioned within the NGT lumen at an offset from the distal end of the NGT when the stop engages the connector on the proximal end of the NGT, and the offset can be between approximately 1-10 mm. The NGT can be adapted for infants and/or is 6.5 French diameter. Illustratively, the light guide is pre-loaded in the NGT when packaged for use.

A method for verifying placement of a nasogastric tube (NGT) in a treatment environment is also provided. A light guide with a light-emitting distal tip is positioned adjacent to a distal end of the NGT. A distal end of the NGT is placed at a location within the patient's anatomy via the nose. A light source is operated to illuminate the light-emitting tip. Light emitted from the tip is visually located through the patient's skin, and based upon the locating, proper placement of the distal end of the NGT is verified with respect to the patient's anatomy. The placement of the distal end of the NGT can be in the stomach of a human infant. Positioning of the distal tip can include engaging a proximal end of the NGT with a stop that limits distal movement of the distal tip of the light guide out of the distal end of the NGT. The light source can be operated to project light approximately radially relative to the distal tip, and the light can be approximately 650 nm. The positioning of the light guide in the NGT can be performed during production so that an assembled version of the NGT and light guide is removed by the user from a package before placement in the patient. After verifying position, the user can proximally remove/withdraw the light guide from the (now-placed) NGT and attaches a food source to the proximal end of the NGT.

### BRIEF DESCRIPTION OF THE DRAWINGS

The invention description below refers to the accompanying drawings, of which:

FIG. 1 is a diagram showing correct placement of an NGT with respect to the esophagus and stomach and a plurality of possible misplacements;

FIGS. 2 and 3 are images showing, respectively, a radiograph and a CT scan used to determine anatomical measurements and specifications for 24 weeks gestational age through 1-year-old infants, and wherein the images demonstrate the distance of the lungs and other organs relative to the stomach;

FIG. 4 is a diagram showing a typical example of the Nose to Earlobe to the Mid-Umbilicus (NEMU) measurement technique to assist in guiding perception of correct placement;

FIG. 5 is a diagram showing a complete fiber optic light guide assembly for use in verifying NGT distal end placement, including an LC connector, rubber stopper, light guide, and radial tip, shown in conjunction with a detachable, handheld light source;

FIG. 6 is a diagram showing the light guide assembly of FIG. 5 installed in an NGT;

FIG. 7 is a further diagram showing the light guide assembly of FIG. 5;

FIG. 8 is a more detailed perspective view of the rubber stopper located on the light guide of FIG. 5;

FIG. 9 is a more detailed perspective view of the robber stopper of FIG. 8 engaging the proximal end connector of the NGT;

FIG. 10 a more detailed perspective view of the radially emitting, distal tip of the light guide of FIG. 5 shown fully inserted into the NGT at a offset distance to avoid organ damage therefrom;

FIG. 11 is a diagram showing the light guide of FIG. 5 initially inserted in the proximal end of the NGT;

FIG. 12 is a diagram showing the light guide of FIG. 5 partially directed into the lumen of the NGT;

FIG. 13 is a diagram showing the light guide of FIG. 5 fully inserted until the stopper meets the proximal end of the NGT; and

FIG. 14 is a flow diagram of a generalized procedure for use of the light guide of FIG. 5 in the insertion of an NGT.

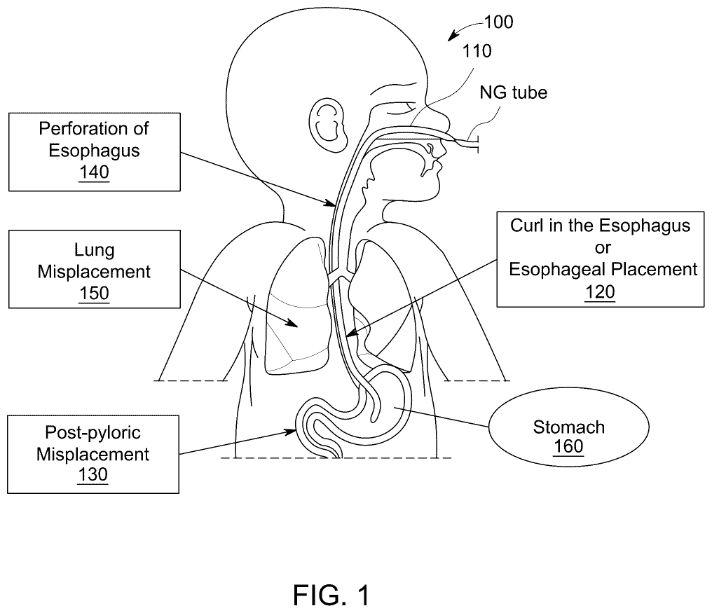

### DETAILED DESCRIPTION

### I. Problem Overview

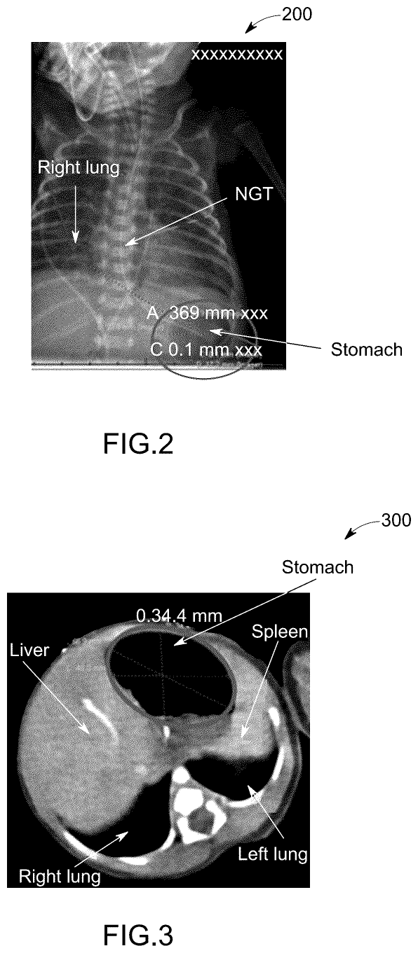

It is recognized that the majority of NGT misplacements are either due to the NGT tube not reaching, or going past the stomach. Thus, in carrying out an NGT insertion procedure, it is imperative to examine neonate anatomy in order to better understand the problem. Given the lack of literature available on neonate anatomy, the size of the stomach and distance to the lung, esophagus, and other organs was determined through analysis of exemplary radiographs and CT scans 200 and 300, shown respectively in FIGS. 2 and 3. Anatomical measurements and ranges used herein are based on the age group found in the ICN, ranging from 24 weeks gestational age up to 1-year-old infants.

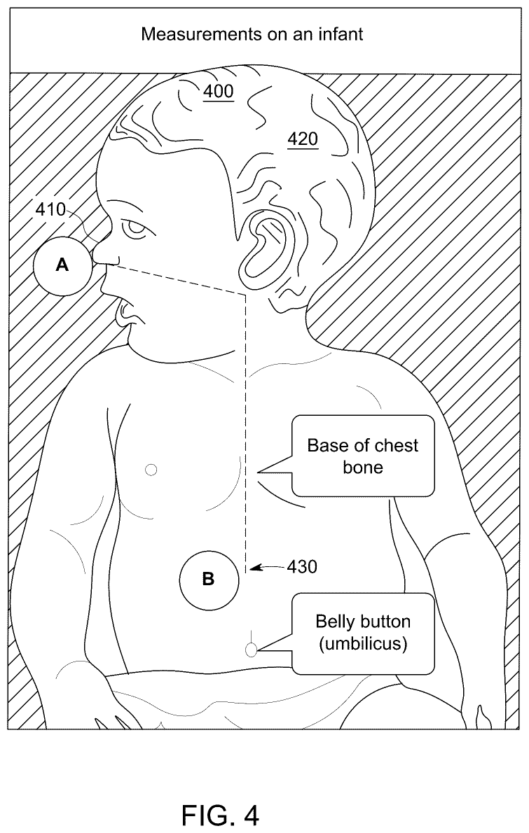

Current NGT verification methods are limited and highly variable across institutions and providers. Current methods include, as shown in FIG. 4, the NEMU technique of measurement from the patient (infant's) 400 nose 410 to ear 420 to mid-umbilicus 430 to estimate the length of NGT required to reach the stomach. The practitioner then inserts the NGT by placing into the patient's nose and pushing it straight back towards the patient's ear, stopping at the expected length (A-B). This only provides a rough guide of placement and is not reliable. As described above, verification techniques for localizing the NGTs distal end, such as X-rays, pH measurements, etc. are unsafe and/or unreliable. Currently, there is no standardized verification method.

### II. NGT Insertion Verification System and Method

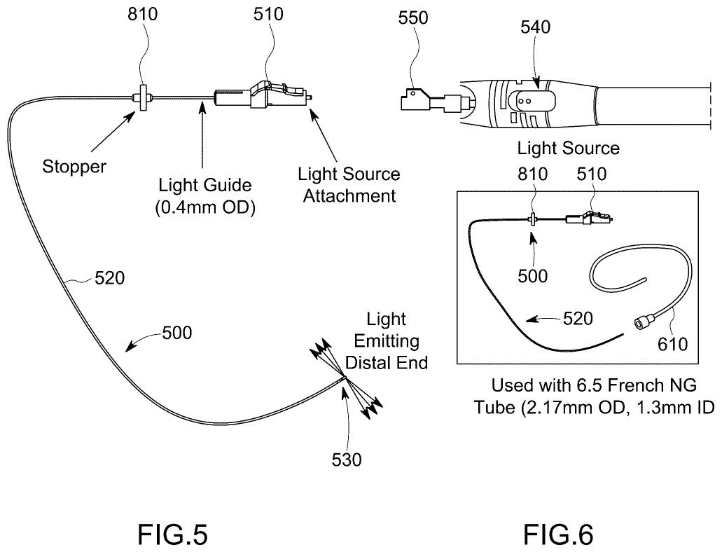

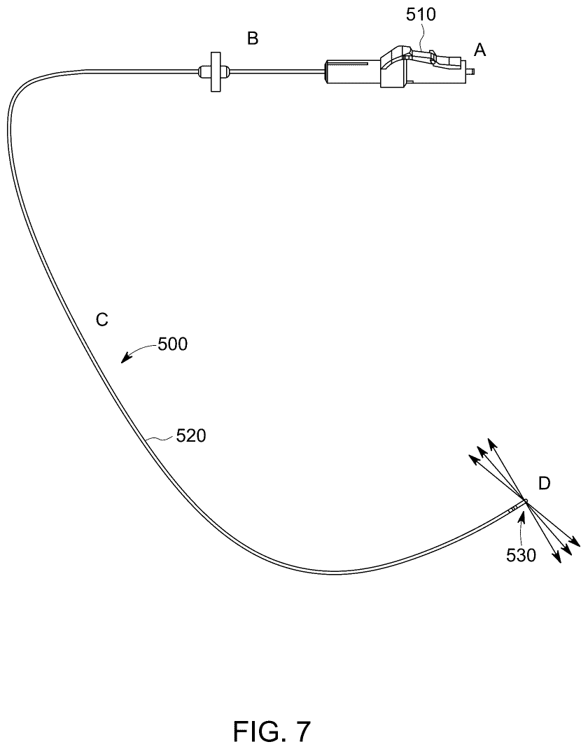

FIGS. 5-10 show a fiber optic assembly 500 for use in localizing and verifying placement of the distal end of an NGT used, for example, for feeding of an infant. The fiber optic assembly 500 consists of a standard LC connector 510 at the proximal end thereof joined to a (e.g.) 400 micron outer diameter (OD) acrylic (fiber optic) light guide 520 having a flexible construction. The distal end of the light guide 520 includes a radially emitting tip 530 (described further below).

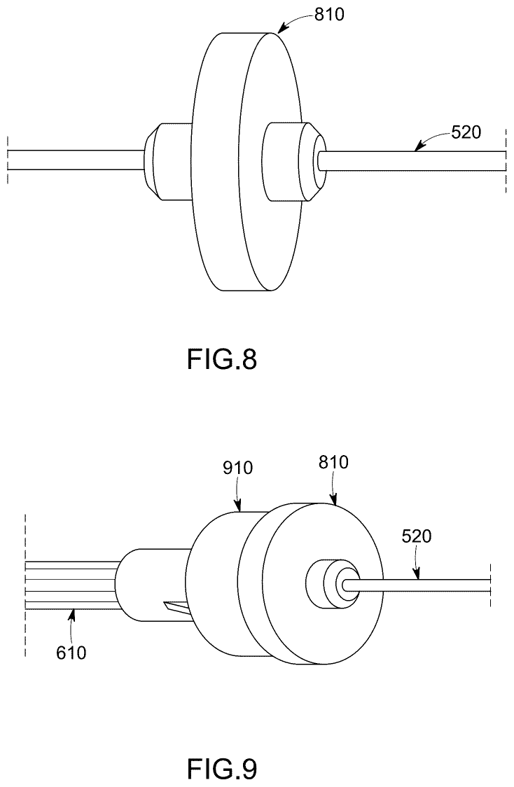

In operation, the light guide is inserted into the lumen of the NGT (610 in FIG. 6) prior to insertion into the patient. With reference particularly to FIGS. 8-10, the light-emitting, distal end 530 of the fiber light guide 520 is located at an offset/standoff approximately 5 mm inboard (proximally) of the distal end 1020 of the NGT 610. The offset distance is highly variable, and is a compromise between accurate localization of the NGT distal end and ensuring that the light guide distal end remains in the lumen of the NGT at all times. This offset is maintained by interference between a rubber stopper (also termed “stop”) fixed to the light guide 520, and the polymer connector 910 (e.g. and Enfit-style connector integral with the NGT) at the proximal end of the NGT 610 (FIG. 9). In an illustrative embodiment, the outer diameter of the light guide 520 is sized to allow insertion into the lumen of a typical 6.5 French NGT. In various embodiments, the offset can be in a range of 1-10 mm, more or less.

The LC connector 510 attached to the proximal end of the light guide 520 in a manner that allows for straightforward connection and disconnection from a commercially available light source 540 during and after NGT placement. The LC connector 510 is this embodiment is constructed from inexpensive polymer, which is sufficient to transmit light down the guide 520. In other embodiments, an alternate connector structure can be used, such as a commercially available (more expensive) threaded aluminum connector.

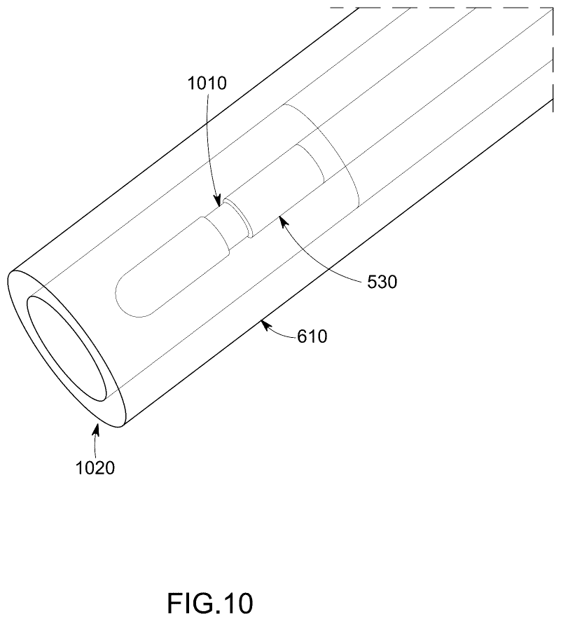

The light guide distal end tip 530 can be a commercially available geometry that, for example, emits light in a 360 degree arc about the longitudinal axis of the light guide 520. See, for example the annular tip emitter 1010 shown in FIG. 10. Such emitting radial tip geometries are commercially available from various vendors and can be constructed in accordance with ordinary skill in the art. This circumferential arrangement ensures that the tip 530 radiates in the direction of a viewer regardless of rotational orientation within the NGT lumen. The distal end 530 of the light guide 520 can define, more particularly, a (e.g.) glass tip which directs light radially outward, perpendicular to the direction the fiber is pointing. This tip geometry leverages the typical orientation of correctly placed NGTs with respect to the stomach, where the NGT rests flush with the wall of the stomach rather than pointed directly at the stomach wall. A radial tip 530 thereby directs light directly toward the outside of the patient's body in this configuration much more effectively than a forward-emitting tip. Advantageously, the tip emits light evenly, radially outwards, and therefore, orientation of the light guide with respect to the NGT does not impact the effectiveness the arrangement.

Note that radial tip light guides that can be employed herein are commercially available from a variety of vendors, such as LaseOptics Corporation of Amherst, NY and Med-Fibers, Inc. of Chandler AZ.

The rubber stopper 810 fixed to the light guide 520 prevents the fiber from extending past the distal end of the NGT 610, preventing any risk of tissue perforation by the light guide. The stopper mechanism 810 simultaneously ensures the location of the light emitting fiber tip is always the same distance from the distal end of the NGT.

In an embodiment, the stopper can be fixed to the light guide so as to define a predetermined length. In alternate embodiments, the light guide can be slidable, while exerting moderate holding friction on the light guide for length adjustability.

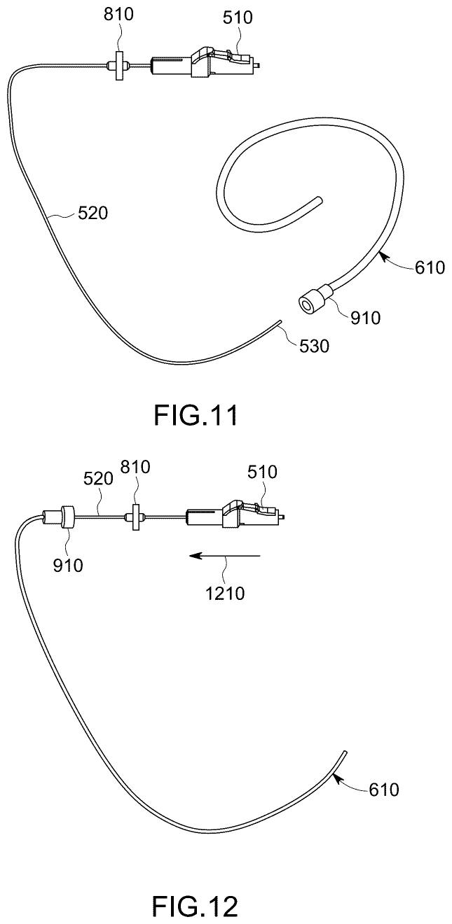

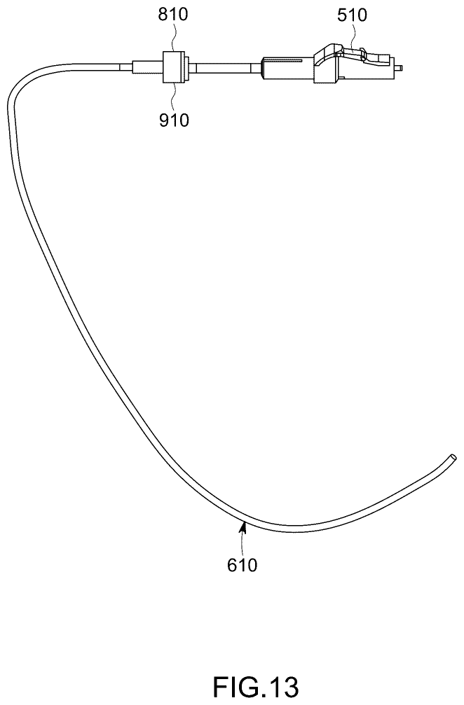

With reference to FIGS. 11-13, the distal tip 530 light guide 520 is shown in preparation to be initially inserted in the proximal end (connector 910) of the NGT 610 (FIG. 11). After insertion, the light guide is driven distally (arrow 1210) down the NGT lumen as shown in FIG. 12 as the stopper 810 approaches the proximal connector 910. Finally, as shown in FIG. 13, the light guide is fully inserted until the stopper 810 engages the proximal end of the NGT 610 and the distal tip is thereby positioned adjacent to the distal end of the NGT at an optimal position to assist proper confirmation of its location in the patient, while avoiding over-extension past the NGT's distal tip.

The light source 540 with an end 550 adapted to removably connect to the LC connector 510 of the light guide 520. More particularly, the light source 540 can be a switchable, battery-operated (or alternatively powered by wall current via a transformer), handheld unit. Visible light is generally preferred as it is readily detected by a user, and the upper wavelength limit of such light is approximately 700 nm. As light penetration in tissue however increases with wavelength, particularly above 600 nm, a wavelength of 650 nm is used for the illustrative light source 640—which provides an appropriate balance of visibility and tissue penetration. More generally, in various embodiments, a wavelength range of approximately 600-700 nm can be used in various embodiments. The power for the 650 nm source 640 can be determined based on ANSI safety standards for maximum permissible exposures for both tissue and naked eyes. Since it is intended to only illuminate the light guide once the assembly is inserted into a patient, tissue exposure is a primary concern. At 650 nm, the maximum permissible exposure for tissue is listed at 200 mW/cm². Based on the geometry of the radially emissive tip 530, the worst-case tissue exposure of 165 mW/cm² is when the loaded NGT is in direct contact with tissue using a 30 mW source. This power level provides ample light penetration through tissue while remaining a safe threshold below the maximum permissible exposure for tissue.

Additionally, maximum permissible ocular exposure at 650 nm is described by the time dependent function 1.8×t0.75×10-3 W*sec/cm². For an expected light activation of 5 seconds during verification, the maximum permissible exposure allows for 6 mW/cm², 20% of the potential exposure if the 30 mW source is shined directly into the naked eye. To minimize the risk of ocular damage going forward, additional safety mechanisms including an interlock can be provided between the fiber assembly and the light source in a manner that the light source can only be activated once the fiber is connected. Such interlock designs can be implemented in accordance with skill in the art.

In various embodiments, the light guide 520 is directed distally through the lumen of the NGT 610 until the stopper 810 engages the proximal connector 910, and then is illuminated to verify placement as the light passes through the patient skin at the relative internal location. In other embodiments, the light guide 520 can comprise a disposable fiber optic assembly that can be loaded (e.g. available as part of the assembled NGT in a sealed package from the manufacturer) into existing NGTs prior to insertion in the patient. Once the loaded NGT is placed in the patient, the proximal end (LC connector 510) of the light guide 520 is connected to a handheld light source 540 to emit light at the NGT's distal end 530 for visual placement verification through the transmission of light through the patient's abdomen. After verification of correct NGT distal end placement in all embodiments, the light guide 520 LC connector 510 is disconnected from the light source 540, withdrawn from the placed NGT 520, and discarded. The NGT can then be connected to a food source.

### III. Workflow Procedure

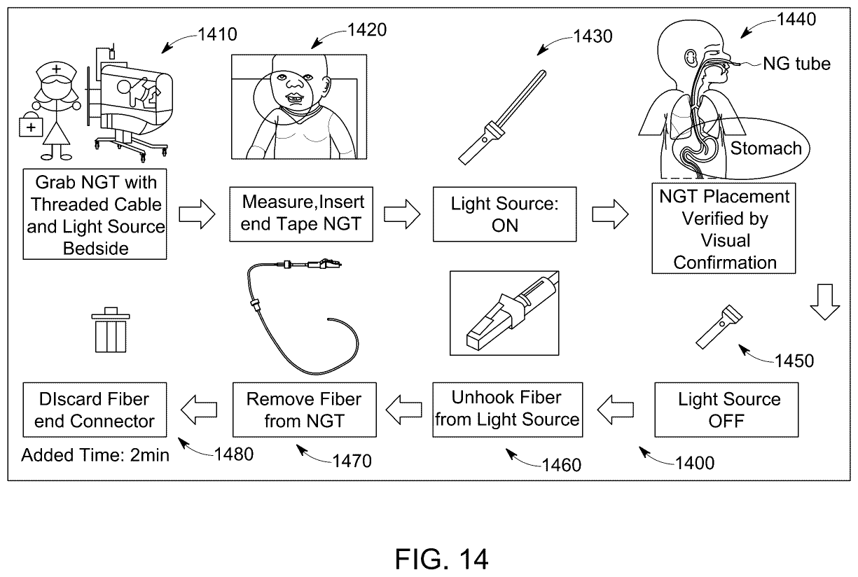

According to an illustrative embodiment, FIG. 14 shows a flow diagram 1400 of a procedure for employing the light guide in association with an NGT. As shown in step 1410, the NGT with loaded light guide is obtained by the practitioner (e.g. a nurse) and a light source that can couple with the light guide LC connector is readied. Next, the NGT and light guide is inserted into the patient until located at the correct distance, and then taped into place in step 1420. The light source is then attached to the light guide in step 1430 and switched on to transmit light at the appropriate wavelength and power. Next, in step 1440, the practitioner visually inspects the patient's body to locate the light emanating through the skin from the distal tip radial emitter. Based upon the location, the proper placement of the NGT is verified—or if the light is not visible or in an improper location, then the NGT is immediately withdrawn, any possible damage is assessed and/or steps 1420-1440 are repeated. Next, in step 1450, the light source is switched off. The light source is then disconnected from the light guide LC connector in step 1460. The light guide is then proximally withdrawn from the now-properly placed NGT in step 1470. The withdrawn light guide can then be disposed of or otherwise discarded in step 1480, and the NGT is ready to receive food input.

### IV. Testing

Testing was performed to determine the efficacy of use of 650 nm light using computer simulations. As such tissue properties were inputted to the simulation program were within its operating range. The simulation studied the impact of skin color on light penetration, as there is an expected decrease in optical light penetration with darker skin tones. Additionally, testing was performed which simulated the anatomy of a neonate, wherein the stomach-body-and skin anatomical proportions were inputted to the simulation program to build meshes that simulated the thickness of each of these tissue types. The absorption and scattering coefficients, the two main properties affecting light penetration through these tissues, were then adjusted to analyze how they affected light penetration in the different regions surrounding the neonate. The light penetration amplitude (in W/mm2) at different detector positions surrounding the outside of the abdomen was the output used to analyze the effect of the tissue properties.

More particularly, varying skin color was simulated through the concentration of melanin in the skin layer of the neonate, where darker skin corresponds to an increase in melanin concentration. The absorption and scattering coefficients for skin tissue ranges from 0.05-1.11 (cm⁻¹) for the absorption coefficient and 2.26-20.96 (cm⁻¹) in the literature corresponding to the range in melanin in different skin types. Therefore, the high, median, and low values for the absorption and scattering coefficients were used to simulate the range in skin tones. Overall, it has been determined that melanin concentration does have an impact on the amount of light penetration, with approximately a 25% decrease in amplitude for W/mm2 from the minimum to the maximum absorption and scattering coefficients for skin in the literature. Therefore, when operating at 30 mW, it is expected to see an attenuation of 7.5 mW as melanin concentration in the skin is maximized. However, the power and wavelength employed herein should be sufficient for use in all skin tones.

A final test involved inserting our prototype into an NGT placed in a dead baby pig. This test allowed us to analyze the effect of different fiber tips on light penetration, quantify the amount of light penetration, and verify our solution concept in realistic anatomy similar to that of neonates.

### V. Conclusion

It should be clear that the above-described system effectively assists in verifying placement of NGTs without adding undue complication of time to the insertion procedure, and while maintaining the current flexibility and size of the NGT, not increasing placement complication rates, and correctly verifying placement to within 1.5 cm at least 95% of the time, with a device lifespan of at least 5 years. Moreover, the system herein provides a quick, reliable arrangement for NGT verification that will help ensure neonate safety and improve a caregivers' confidence during NGT placement. The illustrative system is also less expensive and faster that prior art devices and techniques, while avoiding introduction of any new safety concerns.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, as used herein, various directional and orientational terms (and grammatical variations thereof) such as “vertical”, “horizontal”, “up”, “down”, “bottom”, “top”, “side”, “front”, “rear”, “left”, “right”, “forward”, “rearward”, and the like, are used only as relative conventions and not as absolute orientations with respect to a fixed coordinate system, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances (e.g. 1-2%) of the system. Accordingly, this description is meant to be taken only by way of example, and not to otherwise limit the scope of this invention.

What is claimed is:

1. A system for verification of nasogastric tube (NGT) placement comprising:
a fiber optic light guide having a proximal end with a light source connection and a distal end having a light emitting tip, the light guide having a diameter and a length constructed and arranged for insertion into a lumen of the NGT; and
a stop that prevents the distal end of the light guide from exiting a distal end of the NGT.

2. The system as set forth in claim 1 wherein the stop is adapted to engage a connector on the proximal end of the NGT.

3. The system as set forth in claim 2 wherein the light emitting tip comprises a radial emitter.

4. The system as set forth in claim 2 wherein the light source connection comprises an LC connector that removably engages a handheld, switchable light source.

5. The system as set forth in claim 4 wherein the light source emits light in a range of approximately 600-700 nm.

6. The system as set forth in claim 5 wherein the light source emits light at approximately 650 nm.

7. The system as set forth in claim 6 wherein the light source emits light with an output power of approximately 30W.

8. The system as set forth in claim 2 wherein the stop is located so that the distal end of the light guide is positioned within the NGT lumen at an offset from the distal end of the NGT when the stop engages the connector on the proximal end of the NGT.

9. The system as set forth in claim 8 wherein the offset is between approximately 1-10 mm.

10. The system as set forth in claim 1 wherein the NGT is adapted for infants.

11. The system as set forth in claim 10 wherein the NGT is 6.5 French diameter.

12. The system as set forth in claim 2 wherein the light guide is pre-loaded in the NGT when packaged for use.

13. A method for verifying placement of a nasogastric tube (NGT) comprising the steps of:
positioning a light guide with a light-emitting distal tip adjacent to a distal end of the NGT;
placing a distal end of the NGT at a location within the patient's anatomy via the nose;
operating a light source to illuminate the light-emitting tip;
visually locating light emitted from the tip through the patient's skin; and
based upon the locating, verifying proper placement of the distal end of the NGT with respect to the patient's anatomy.

14. The method as set forth in claim 13 wherein the step of placing includes placing the distal end into a stomach of a human infant.

15. The method as set forth in claim 13 wherein the step of positioning includes engaging a proximal end of the NGT with a stop that limits distal movement of the distal tip of the light guide out of the distal end of the NGT.

16. The method as set forth in claim 1 wherein the step of operating the light source includes projecting light approximately radially relative to the distal tip.

17. The method as set forth in claim 16 wherein the step of projecting includes projecting light at approximately 650 nm.

18. The method as set forth in claim 13 wherein the step of positioning is performed during production and further comprising removing an assembled version of the NGT and light guide from a package for use in the step of placing.

19. The method as set forth in claim 13, further comprising, after the step of verifying, proximally removing the light guide from the NGT and attaching a food source to the proximal end of the NGT.
